(19) United States
(12) Patent Application Publication
(10) Pub. No.: US 2026/0240750 A1
(43) Pub. Date: Aug. 20, 2026

(54) AN ESTER PRODUCT OF POLYGLYCEROL

(71) Applicant: Fine Organic Industries Limited, Mumbai (IN)

(72) Inventors: Rahul PATIL, Mumbai (IN); Shriniwas MACHHINDRANATH SAWANT, Mumbai (IN); Mayukh WARAWDEKAR, Navi Mumbai (IN); Konstantinos LAHANAS, Paramus, NJ (US)

(21) Appl. No.: 18/876,575

(22) PCT Filed: Jun. 27, 2023
(86) PCT No.: PCT/IN2023/050626

§ 371 Date: Dec. 18, 2024

(30) Foreign Application Priority Data
Jul. 1, 2022 (IN) 202221037949

(51) Int. Cl. A61K 8/86; A61Q 5/02; C08G 65/48

(52) U.S. Cl. CPC A61K 8/86; A61Q 5/02; C08G 65/48; A61K 2800/10; A61K 2800/43; A61K 2800/524

(57) ABSTRACT

An ester product of polyglycerol with C8 and C10 fatty acids & lactic acid; wherein the polyglycerol is with an average polymeric degree of 3 to 10; and its use in preparing stable personal care compositions.

The present invention relates to an ester product of polyglycerol with C₈ and C₁₀ fatty acids & lactic acid and its use in the preparation of stable personal care compositions.

### BACKGROUND OF THE INVENTION

United States Patent Application Publication number 2017/0071836 (applicant M/w Henkel AG, referred to herein as '836) exemplifies use of fatty acyl lactylates as cosmetic cleansing agents. However, the present invention prepares a polyglycerol ester product and its use in stabilizing personal care compositions.

Chinese Patent application No. 106588648 discloses preparation of polyglycerol esters wherein the polyglycerol is a tetramer. However, it suggests use of the polyglycerol esters as moisturizers. However, the present invention prepares a polyglycerol ester product and its use in stabilizing personal care compositions.

### OBJECT OF THE INVENTION

The object of the present invention is to provide an ester product of polyglycerol with C₈ and C₁₀ fatty acids & lactic acid for the preparation of stable personal care compositions.

Another object of the present invention is to provide stable personal care compositions comprising an ester product of polyglycerol with C₈ and C₁₀ fatty acids & lactic acid which facilitates wetting, dispersion and reduced settling of hydrophobic pigments during storage.

### SUMMARY OF THE INVENTION

An ester product of polyglycerol with C₈ and C₁₀ fatty acids & lactic acid; wherein the polyglycerol is with an average polymeric degree of 3 to 10.

An ester product of polyglycerol with C₈ and C₁₀ fatty acids & lactic acid with ester selected from glycerol with C₈ and C₁₀ fatty acids and/or polyglycerol with lactic acid and C₁₂ fatty acid in the preparation of stable personal care compositions.

Additionally, the personal care compositions comprising the ester product of the present invention will be free of preservatives.

### DESCRIPTION OF THE INVENTION

Personal care compositions with mild preservatives are in demand by consumers.

It is known that sulfates in personal care compositions may damage cells and strip away natural oils and hair proteins & ethanolamide based surfactants may comprise nitroso compounds which are considered harmful when in contact with skin.

In view of the same we have developed a personal care composition which is stable and mild. The personal care composition of the present invention is user friendly as it is preservative free, sulfate free, amide free, ethylene oxide free, silicone free, paraben free and formaldehyde free.

According to one embodiment of the present invention is an ester product of polyglycerol with C₈ and C₁₀ fatty acids & lactic acid; wherein the polyglycerol is with an average polymeric degree of 3 to 10.

Typically, the polyglycerol comprises 20 to 40 weight % of glycerol.

The combined C₈ and C₁₀ fatty acids will be 5-15 parts by weight of the polyglycerol.

The lactic acid is 3 to 8 parts by weight of the polyglycerol.

The ester product of polyglycerol with C₈ and C₁₀ fatty acids & lactic acid; wherein the polyglycerol is with an average polymeric degree of 3 to 10 facilitates dispersion of hydrophobic pigments by improving wettability in personal care compositions.

A good wetting agent first and foremost gives good particle separation in the bulk. As a consequence, intra-particle agglomeration and settling are reduced or eliminated. This would typically be seen on stability studies of the finished product such as personal care compositions either at ambient or accelerated conditions as a liquid separation, solid agglomerates, or a precipitate.

Improved dispersion of hydrophobic pigments inhibits settling of hydrophobic pigments leading to stable personal care compositions.

The ester product of polyglycerol with C₈ and C₁₀ fatty acids & lactic acid; wherein the polyglycerol is with an average polymeric degree of 3 to 10 disperses hydrophobic pigments in 40 to 80% concentration.

A personal care composition comprising
(a) 0.1 to 5 weight % of an ester product of polyglycerol with C₈ and C₁₀ fatty acids & lactic acid; wherein the polyglycerol is with an average polymeric degree of 3 to 10;
(b) at least one hydrophobic pigment;
(c) at least one excipient; and
(d) water;
wherein the weight % is with regards to the composition and the composition is physically stable at ambient conditions.

Physical stability is measured visually in the form of liquid separation, solid agglomerates, or a precipitate.

The personal care composition of the present invention is selected from shampoo, lotions, creams, veggie wash, different personal care preparation scalp cleanser, lotions, creams, veggie wash, different personal care preparation, hair cleanser, shower preparations, shower gels, foam baths, gels, body wash, facial cleanser, face wash, makeup remover, cleansing wipe, sunscreen, foundation and the like.

The excipients used in the personal care composition of the present invention are selected from anionic, cationic, zwiterrionic, nonionic, amphoteric surfactants; superfatting agents, stabilizers, biogenic active ingredients, humectants, preservatives, pearlizing agents, dyes and pigments, fragrances, solvents, opacifiers, further thickeners and dispersants, protein derivatives such as gelatin, collagen hydrolysates, polypeptides, fatty alcohols, odor masking agents and enzymes.

The ester product of polyglycerol with C₈ and C₁₀ fatty acids & lactic acid; wherein the polyglycerol is with an average polymeric degree of 3 to 10 provides antimicrobial properties to the personal care composition effective in inhibiting growth of positive and negative bacteria at 0.05 to 5.0% concentration.

The bacteria may be selected from Staphylococcus aureus, Bacillus subtilis, E. Coli, Pseudomonas aeruginosa, Salmonella enteric, Propionebacterium acne, Malassezia furfur, Streptococcus mutans, Lactobacillis acidophilus, Candida albicans, Aspergillus niger and the like.

The personal care composition may further comprise ester selected from glycerol with C₈ and C₁₀ fatty acids and/or polyglycerol with lactic acid and C₁₂ fatty acid.

### Definition of Terms

### Dropping Point

The dropping point is the temperature at which a substance passes from a semi-solid to a liquid state.

### Pour Point

The pour point of a liquid is the lowest temperature at which it becomes semi solid. This is where the liquid loses its flow characteristics.

### Acid Value

Acid value (or neutralization number or acid number or acidity) is the mass of potassium hydroxide (KOH) in milligrams that is required to neutralize one gram of chemical substance. The acid number is a measure of the number of carboxylic acid groups in a chemical compound, such as a fatty acid.

### Saponification Value

Saponification value or saponification number (SV or SN) represents the number of milligrams of potassium hydroxide (KOH) required to saponify one gram of fat under the conditions specified.

### Hydroxyl Value

The hydroxyl value of a substance is the amount, in milligrams, of potassium hydroxide required to neutralize any acid when combined by acylation in 1 g of the substance under examination.

### Iodine Value

The iodine value (or iodine adsorption value or iodine number or iodine index, commonly abbreviated as IV) is the mass of iodine in grams that is consumed by 100 grams of a chemical substance. Iodine value is used to determine the amount of unsaturation in fats, oils and waxes.

The following examples illustrate preferred embodiments in accordance with the present invention without limiting the scope of the invention.

### EXAMPLES

### Example 1

### Raw Materials:

| Sr no | Material | Qty (gm) |
|---|---|---|
| 1 | Polyglycerol(crude) | 396.0 |
| 2 | Capric-Caprylic acid | 79.2 |
| 3 | Lauric Acid | 26.4 |
| 4 | Lactic acid | 29.6 |
| 5 | Catalyst | 0.520 |
| 6 | NaOH | 0.052 |

Place in a heating mantle, a four necked round bottom flask fitted with a mechanical stirrer, temperature sensor, gas sparger and a packed column, add Polyglycerol. Under stirring introduce Nitrogen gas to maintain inert atmosphere, heat to 75° C.

Add sequentially Capric-Caprylic acid, Lauric acid. Lactic acid, VPA and KOH and raise the temp to 180° C. ensuring that the vapour temp at the top of the packed column is ≤99-100° C. Maintain at this temp till the reaction water steam reduces and raise the reaction temperature to 200° C. ensuring that the vapour temp does not exceed 99-100° C.

Withdraw reaction sample after 3 hrs. to measure Acid Value to monitor the progress of the reaction. Continue the reaction till A.V. reaches <1 mg KOH/g, cool the reaction mass and neutralize the same at 90° C. with 50:50 NaOH-Water. Continue stirring at this temperature for ~30 minutes and filter the reaction mass. The ester of the present invention, Finester 2504, is used for the examples below.

Typical characteristic values of Finester 2504 are as follows—

| Sr. No. | Particulars | Finester 2504 |
|---|---|---|
| 1 | Dropping Point | <−5° C. |
| 2 | Acid Value | 2.0 Max |
| 3 | Sap Value | 105 |
| 4 | Hydroxyl Value | 900 |
| 5 | Iodine Value | <1 |

### Test Methods Used

### Pour Point: Manual Method

The specimen is cooled inside a cooling bath to allow the formation of crystals. At about 9° C. above the expected pour point, and for every 3° C. after, the test jar is removed and tilted to check for surface movement. When the specimen does not flow when tilted, the jar is held horizontally for 5 seconds. If it does not flow, 3° C. is added to the corresponding temperature, since this is the last measurement when flow was observed, and the result is the pour point temperature.

An approximate range of pour point can be observed from the specimen's upper and lower pour point.

This is helpful with products as it gives a temperature window depending on its thermal history. Within this temperature range, the sample may appear liquid or solid. This happens because the crystals form more readily when it has been heated within the past 24 hrs and contributes to the lower pour point.

The upper pour point is measured by pouring the test sample directly into a test jar. The sample is then cooled and inspected for the pour point.
Acid Value: A.O.C.S. Cd 3d-63(09)
Saponification Value: A.O.C.S. Cd 3-25(09)
Hydroxyl Value: A.O.C.S. Cd 13-60(16)
Iodine Value: A.O.C.S. Tg 1a-64(09)

### Example 2: Sulfate Free Conditioning Shampoo

| Ingredient | % w/w |
|---|---|
| Deionized Water | 56.75 |
| Dermofeel PA-12 (sodium phytate) | 0.10 |
| Amaze XT Dehydoxanthan Gum | 2.00 |
| Iselux LQ-CLR-SB Sodium Lauroyl Methyl Isethionate | 10.00 |
| Chembetain CGF Cocamidopropyl betaine | 8.00 |
| Finester 2009 Polyglycerin lauroyl lactylate | 17.00 |
| Finester 2199 Sodium lauroyl lactylate | 3.00 |
| D-Panthenol | 1.00 |
| Finester 2504 Ester of Example 1 | 2.00 |
| Citric Acid Anh. | 0.15 |
| | 100 |

### Preparation of the Shampoo:

1. In main vessel add Deionized Water. Dissolve Dermofeel PA-12.
2. Add remaining ingredients except Amaze XT and Diocide. Heat to 80 C.
3. Sprinkle in Amaze XT with moderate mixing, allowing air to escape. When all is added mix faster with mixer deep and without sucking in air.
4. Hold at 80 C for one hour or until thick and uniform. Cool to 35 C. Add Finester 2504. QS. Mix until uniform

### Test Values

| | |
|---|---|
| pH | 4.92 |
| Viscosity (cps) Brookefield DVLVII+, 6 rpm | 606112 |

### Example 3: Visual Testing for Stability

Clear homogenous mass.

No sedimentation on standing.

### Example 4: Microbial Testing

TABLE 1
Summary of Determination of Minimum Inhibitory Concentration/Minimum
Bactericidal Concentration using Broth Dilution Tube Method

| Sample Code | Test Organism | MIC Value | MBC Value |
|---|---|---|---|
| Product - Finester 2504 | Enterococcus hirae ATCC 10541 | Inconclusive* | 12.5% |
| Ester of Example 1 | Salmonella enterica ATCC 10708 | Inconclusive* | >25% |
| Batch no- 0466012231 | Pseudomonas aeruginosa ATCC 15442 | Inconclusive* | >25% |
| | candida albicans ATCC 10231 | Inconclusive* | 12.5% |
| | Aspergillus brasiliensis ATCC 16404 | Inconclusive* | 12.5% |
| | Streptococcus mutans ATCC 25175 | Inconclusive* | <0.20% |
| | Malassezia furfur ATCC 14521 | Inconclusive* | 25% |

Key:
*Inconclusive as the presence or absence of growth could not be identified visually; due to inherent turbidity of the test sample.

We claim:

1. An ester product of polyglycerol with C₈ and C₁₀ fatty acids & lactic acid; wherein the polyglycerol is with an average polymeric degree of 3 to 10.

2. An ester product of polyglycerol as claimed in claim 1 wherein the polyglycerol comprises 20 to 40 weight % of glycerol.

3. An ester product of polyglycerol as claimed in claim 1 wherein the combined C₈ and C₁₀ fatty acids are 5-15 parts by weight of the polyglycerol.

4. An ester product of polyglycerol as claimed in claim 1 wherein the lactic acid is 3 to 8 parts by weight of the polyglycerol.

5. An ester product of polyglycerol as claimed in claim 1 wherein the ester product facilitates dispersion of hydrophobic pigments in personal care compositions.

6. An ester product of polyglycerol as claimed in claim 1 wherein the ester product inhibits settling of hydrophobic pigments in personal care compositions.

7. An ester product of polyglycerol as claimed in claim 1 wherein the ester product disperses hydrophobic pigments in 40 to 80% concentration.

8. A personal care composition comprising
(a) 0.1 to 5 weight % of an ester product as claimed in claim 1;
(b) at least one hydrophobic pigment;
(c) at least one excipient; and
(d) water;
wherein the weight % is with regards to the composition and the composition is physically stable at ambient conditions.

9. A personal care composition as claimed in claim 8 wherein the composition has antimicrobial properties effective in inhibiting growth of positive and negative bacteria at 0.05 to 5.0% concentration.

10. A personal care composition as claimed in claim 8 wherein the composition further comprises ester selected from glycerol with C₈ and C₁₀ fatty acids and/or polyglycerol with lactic acid and C₁₂ fatty acid.

11. A personal care composition as claimed in claim 8 selected from shampoo, scalp cleanser, hair cleanser, shower preparations, shower gels, foam baths, gels, body wash, facial cleanser, face wash, makeup remover, cleansing wipe, sunscreen, foundation and the like.

12. A personal care composition as claimed in claim 8 wherein the excipients are selected from anionic, cationic, zwiterrionic, nonionic, amphoteric surfactants; superfatting agents, stabilizers, biogenic active ingredients, humectants, preservatives, pearlizing agents, dyes and pigments, fragrances, solvents, opacifiers, further thickeners and dispersants, protein derivatives such as gelatin, collagen hydrolysates, polypeptides, fatty alcohols, odor masking agents and enzymes.
